(19) United States
(12) Patent Application Publication
(10) Pub. No.: US 2011/0117154 A1
(43) Pub. Date: May 19, 2011

(54) USE OF RIFALAZIL TO TREAT COLONIC DISORDERS

(75) Inventor: Chalom Sayada, Luxembourg City (LU)

(73) Assignee: ACTIVBIOTICS PHARMA, LLC, Tucker, GA (US)

(21) Appl. No.: 13/002,702

(22) PCT Filed: Jun. 30, 2009
(86) PCT No.: PCT/US09/49288

§ 371 Date: Jan. 20, 2011

Related U.S. Application Data
(60) Provisional application No. 61/078,443, filed on Jul. 7, 2008.

(51) Int. Cl. A61K 9/14; A61K 31/5383; A61K 38/14; A61P 31/04; A61P 1/00

(52) U.S. Cl. 424/405; 514/229.5; 514/3.1

(57) ABSTRACT

Methods for treating bacterial infections in the colon, and colonic disorders caused by bacterial infection, using a poorly absorbable form of Rifalazil, are described. Compositions for oral administration, and colonic delivery, of a non- micro-granulated Rifalazil formulation, are also described. Rifalazil is delivered in a form which is poorly absorbed in the gut after oral dosing, and the vast majority of the orally-dosed Rifalazil is not absorbed in the gut. Accordingly, the antibacterial potency in the colonic flora will be enhanced, while absorption and systemic circulation will be reduced, thus reducing potential adverse events.

### FIELD OF THE INVENTION

The invention is generally directed to the use of Rifalazil to treat bacterial infections in the gastrointestinal tract, while maintaining a minimal absorption in the systemic circulation, and minimizing adverse events from the antibiotic administration.

### BACKGROUND OF THE INVENTION

The intestinal bacterial flora has a significant role in the etiopathogenesis of the intestinal inflammatory diseases such as Crohn's disease, and the disorders tend to be localized in areas with high bacteria concentrations Animal models have shown that spontaneous colitis does not develop if a “germ-free” condition is maintained (Blumberg R. S., in Curr. Opin. Immunol., 1999, 11(6), 648-56), and inflammation of the intestinal mucous membrane develops after the contact with fecal material (Harper P. H., in Gut 1985, 26(3), 279-84).

Antibiotics are usually used to decrease the growth of the luminal bacteria; to decrease the inflammatory state sustained as a result of the bacterial growth; to reduce symptoms of the acute phase of the disease, e.g., diarrhea, intestinal pain and meteorism; and to prevent and to cure septic complications, such as abscesses, fistulas and toxic state.

Most antibiotics are systemically absorbed. Vancomycin (oral formulation), Metronidazole and ciprofloxacin are often used to treat colonic infections, often at relatively high doses for a relatively long period of time. However, because these drugs have a high systemic bioavailability, and/or because they have broad anti-bacterial spectrum activity causing alterations of the normal colonic microflora, and/or because these drugs can select for bacterial resistance which can systemically cause septicemia causing deaths (Vancomycin Resistant Enteroccocis, VREs), they are associated with a high incidence of side effects, including exacerbation of the bacterial infections, metallic taste, gastric intolerance, nausea, glossitis, cephalea, vertigo, ataxia, convulsion, neurotoxicity, and peripheral neuropathy.

It would therefore be advantageous to treat colonic disorders with antibiotics that are highly active against a wide range of undesired bacteria, with limited antibacterial activity against normal colonic bacteria, with limited drug resistance selection and which are also poorly bioavailable, to minimize systemic side effects, even on long term dosing at high concentrations.

Rifamycin antibiotics have been proposed for use in treating a variety of disorders. Rifalazil is a synthetic antibiotic designed to modify the parent compound, rifamycin. Compared to other antibiotics in the rifamycin class, it has extremely high antibacterial activity. However, while it has a broad spectrum of antibacterial action covering Gram-positive and Gram-negative organisms, both aerobes and anaerobes, it also has low solubility, which hinders its ability to be administered systemically.

There have been several methods proposed to overcome the solubility issues associated with Rifalazil. For example, U.S. Pat. No. 5,547,683 is directed to a method for producing a microgranulated particle form of Rifalazil, and U.S. patent application Ser. No. 10/453,155 discloses intravenous compositions including Rifalazil.

While these systemic formulations can be advantageous for treating certain disorders, it would be advantageous to provide new uses for Rifalazil that take advantage of its low solubility, as well as to provide new pharmaceutical compositions for delivering Rifalazil in a manner in which one can take advantage of its low solubility. The present invention provides such compositions and uses.

### SUMMARY OF THE INVENTION

Methods for treating bacterial infections in the colon, and colonic disorders caused by bacterial infection, using a poorly absorbable form of Rifalazil, are disclosed. Compositions for oral administration, and colonic delivery, of a non-microgranulated Rifalazil formulation, are also disclosed.

Rifalazil is delivered in a form that is poorly absorbed in the gut after oral dosing, and the vast majority of the orally-dosed Rifalazil is not absorbed in the gut. Accordingly, the antibacterial potency in the colonic floral environment will be enhanced, while absorption and systemic circulation will be reduced, thus reducing potential adverse events and maintaining a minimal amount of Rifalazil absorbed which will allow the unabsorbed Rifalazil to re-circulate in the colon to enable longer term antibacterial effect and prevent potential relapses or bacterial reinfections.

The compositions predominantly include Rifalazil, along with one or more pharmaceutically acceptable excipients and carriers. While the invention is described herein with particular reference to Rafalazil, it is to be appreciated that the invention may be carried out with Rifalazil derivatives as the active component of the therapeutic composition. The compositions can include a minor amount, e.g., less than about 15% by weight of ingredients, to provide minimal solubility to the Rifalazil. In one embodiment, a portion of the rifalazil is delivered systemically, and eliminated through the colon, whereby it is available to treat any of the bacterial infection not treated by the initially-delivered amount of poorly-absorbed Rifalazil.

In another embodiment, the invention is directed to Rifalazil-containing tablet formulations for oral administration. Using these formulations, one can deliver Rifalazil to the colon in amounts sufficient to treat diseases brought on by bacterial infection of the colon. In a preferred aspect of this embodiment, the formulations are administered orally, but administer a substantial portion of the Rifalazil to the colon.

Such pharmaceutical formulations can be in the form of microgranules, made gastro-resistant by coating them with a polymer, which polymer is insoluble at pH values between 1.5 and 4.0 (i.e., the pH of the stomach), and could be partially or entirely soluble at pH values between 5.0 and 7.5 (i.e., the pH of the colon).

The methods can be used to treat a subject having antibiotic-associated bacterial diarrhea, or a Clostridium (C.) difficile infection, or to prevent such a disease or infection in the subject.

In one embodiment, the methods involve administering a composition comprising a combination of Rifalazil and vancomycin. The vancomycin can be suitable for oral or intravenous administration. The unit dosages for Rifalazil can range from 0.01 to 1000 mg (e.g., between 1 and 1000 mg, or between 1 and 100 mg, or between 1 and 25 mg, or between 1 and 5 mg), or reside in any other therapeutic range, and the unit dosages for vancomycin can range from 125 to 2000 mg, or from 500 to 2000 mg or from 750 to 1500 mg, or reside in any other suitable therapeutic range.

Other features, aspects and embodiments of the invention will be more fully apparent from the ensuing disclosure and appended claims.

### BRIEF DESCRIPTION OF THE FIGURES

FIG. 1 shows the survival of hamsters with no infection (□), and hamsters infected with C. difficile and treated with no drug (O), with vancomycin (Δ), or with Rifalazil (*).

### DETAILED DESCRIPTION OF THE INVENTION

The invention described herein relates to the discovery that Rifalazil, administered in a poorly-soluble form, alone or in combination with one or more additional antibiotics, anti-toxins, and the like, can be effective to treat a subject having antibiotic-associated bacterial diarrhea, an infection of Clostridium (C.) difficile, or other disorders associated with infection in the gastrointestinal tract, such as the colon.

The present invention in various specific embodiments utilizes Rifalazil particles, at a size in which the particles are poorly absorbed, to treat colonic diseases such as CDAD, Staphylococci's associated diarrhea, Chrohn's disease, Colitis, intestinal bowel diseases, and the like.

Using the non-microgranulated formulation, the Rifalazil will be poorly absorbed in the gut after oral dosing, and the vast majority of the oral dosed rifalazil is not absorbed in the gut. This enhances the anti-bacterial potency in the colon flora, while reducing absorption and systemic circulation, thereby reducing potential adverse events.

The present invention will be better understood with reference to the following detailed description, and with respect to the following definitions.

### DEFINITIONS

As used herein, poorly soluble means a classification of a therapeutic agent in the Biopharmaceutical Classification System (BCS) of Class III or Class IV. In general, therapeutic agents having a solubility below 0.1 mg/mL present significant solubilization difficulties, and even compounds with solubilities below 10 mg/mL may present solubilization issues during their formulation.

“Antibiotic-associated bacterial diarrhea” means a condition in which antibiotic therapy disturbs the balance of the microbial flora of the gut, allowing pathogenic organisms such as C. difficile and other organisms which cause diarrhea to flourish. Antibiotic-associated bacterial diarrhea specifically includes such conditions as C. difficile associated diarrhea (CDAD) and pseudomembranous colitis.

The term “an effective amount” refers to the amount of Rifalazil, alone or in combination with one or more additional antibiotics, needed to eradicate the C. difficile or other bacterial infection from the subject, or to prevent an infection of C. difficile or other bacterial infection, as determined by a diagnostic test that detects C. difficile or other infection.

One example of a diagnostic test is the use of a commercially available enzyme-linked immunoassay (ELISA; Immunocard; Meridian Diagnostics, Inc., Cincinnati, Ohio) to detect the presence of C. difficile toxin A protein in cecal content extracts. Another example of a diagnostic test is the use of a cytotoxicity assay using human fibroblast cells (Toxi-Titer; Bartels, Inc., Issaquah, Wash.) to detect the presence of C. difficile toxin B. Both of these examples can be found in McVay and Rolfe (Antimicrobial Agents and Chemo. 44:2254-2258, 2000).

An “effective amount” can also mean the amount of Rifalazil, alone or in combination with one or more additional antibiotics, required to reduce the symptoms of a C. difficile-associated disease in a subject or animal model. The symptoms of the disease include diarrhea, weight loss, lethargy, and ruffled fur in specific animal models. Standard assays present in the art can be used to measure the symptoms of disease (for examples of assays see Boon and Beale, Drugs Suppl. 5:57-63, 1985 and McVay and Rolfe, supra).

An “effective amount” of Rifalazil, alone or in combination with one or more additional antibiotics, reduces the symptoms of C. difficile-associated disease in a subject by 20%, preferably, 30% or 40%, more preferably, 50% or 60%, and most preferably, 70%, 80%, 90%, or more, as compared to an untreated subject.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

The term “lower gastrointestinal tract” means the lower part of the small intestine (ileum) and the colon.

The term “enteric coating” means a coating surrounding a core, the solubility of the coating being dependent on the pH in such a manner that it substantially prevents the release of a drug in the stomach, but permits the release of the drug at some stage after the formulation has emptied from the stomach. The term “pH-sensitive enteric polymer” means a polymer the solubility of which is dependent on the pH so that it is insoluble in the gastric juice but dissolves at some stage after the formulation has emptied from the stomach.

By “subject” is meant any warm-blooded animal including but not limited to a human, cow, horse, pig, sheep, goat, bird, mouse, rat, dog, cat, monkey, baboon, or the like. It is most preferred that the subject be a human.

### I. Rifalazil

As used herein, “Rifalazil” refers to 3′-hydroxy-5′-(4-isobutyl-1-piperazinyl) benzoxazinorifamycin, also known as KRM-1648 or ABI1648. Methods of making rifalazil and microgranulated formulations thereof are described in U.S. Pat. Nos. 4,983,602 and 5,547,683, respectively. The invention as previously discussed contemplates the use of Rifalazil derivatives that are similar or superior in therapeutic effect to Rifalazil.

Rifalazil is a synthetic antibiotic designed to modify the parent compound, rifamycin. Compared to other antibiotics in the rifamycin class, it has extremely high antibacterial activity. However, while it has a broad spectrum of antibacterial action covering Gram-positive and Gram-negative organisms, both aerobes and anaerobes, it also has low solubility.

Particle Size Range

The Rifalazil used in the invention described herein can be in the form of crystals or in amorphous form, is poorly absorbed, and is not very soluble in a variety of commonly used FDA-approved liquid formulation ingredients. As used here, the term “Rifalazil in poorly dissolvable form” means that the particle size of the Rifalazil is greater than about 10 μm, preferably great than about 50 μm, and, most preferably, greater than about 100 μm. Rifalazil particles of this size range are believed to have limited potential absorption and solubility. Various salt forms of Rifalazil also can be used in the broad practice of the present invention.

### II. Pharmaceutical Compositions

Ideally, the Rifalazil is administered in a composition that is administered orally, but which delivers the Rifalazil to the colon. Representative drug delivery formulations for oral administration, and colonic delivery, are described, for example, in U.S. Pat. No. 5,958,873 and PCT WO/2006/094737, the contents of which are hereby incorporated by reference.

The dosage of Rifalazil in various specific embodiments can range from about 0.01 to 1000 mg., although any specific dosage that is advantageous in a given application can be employed. The dosage of Rifalazil in various embodiments can be any suitable amount, e.g., about 1 to 1000 mg (desirably about 1 to 100 mg, more desirably about 1 to 50 mg, and even more desirably about 1 to 25 mg). The Rifalazil may be given daily (e.g., once, or twice daily) or less frequently (e.g., once every other day, once or twice weekly, or twice monthly), or in any other dosing regimen that provides therapeutic benefit. The administration of Rifalazil can be by any suitable means that results in an effective amount of the compound reaching the target region, for example, the colon.

The compound may be contained in any appropriate amount in any suitable carrier substance, and is generally present in an amount of 1-95% by weight of the total weight of the composition. In one embodiment, the composition is provided in a dosage form that is suitable for oral administration, e.g., a tablet, capsule, pill, powder, granulate, suspension, emulsion, solution, or gel.

The pharmaceutical composition can generally be formulated according to conventional pharmaceutical practice (see, e.g., Remington: The Science and Practice of Pharmacy (20th ed.), ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, N.Y.).

The pharmaceutical compositions used to deliver the Rifalazil can be formulated to release Rifalazil at a predetermined time period, or set of criteria (i.e., upon reaching a certain pH) so that the Rifalazil is administered to the colon, or immediately prior to the colon.

When controlled release formulations are used, they are preferably a) formulations that after a predetermined lag time create a substantially constant concentration of the drug within the colon over an extended period of time, b) formulations that localize drug action by, e.g., spatial placement of a controlled release composition adjacent to or in the colon; or (c) formulations that target drug action by using carriers, coatings, or excipients that degrade in the colon, but not elsewhere in the gastrointestinal tract.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, preferably to delay disintegration and absorption in the gastrointestinal tract until the tablets reach the colon. The coating can be adapted to not release the Rifalazil until after passage through the stomach, for example, by using an enteric coating (e.g., a pH-sensitive enteric polymer). Advantageously, a substantial amount of the drug is released in the lower gastrointestinal tract, such as the colon or immediately prior to the colon.

The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

The solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, supra.

Formulations for oral use may also be presented as hard gelatin capsules wherein the active ingredient is mixed with an inert solid diluent (e.g., potato starch, lactose, microcrystalline cellulose, calcium carbonate, calcium phosphate or kaolin). Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may be constructed to release the active drug by controlling the dissolution and/or the diffusion of the active drug substance.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. Additional examples include the formulations listed on the following websites: http://www.advancispharm.com/, http://www.intecpharma.com/, and www.depomedinc.com/

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl inethacrylate, polyvinyl chloride, polyethylene, and/or halogenated fluorocarbon.

Representative Formulations for Oral Administration and Colonic Delivery

To maximize the therapeutic efficacy of Rifalazil in the treatment of bowel diseases, it is advantageous to provide oral administration, and colonic delivery, of the Rifalizal. In one embodiment, the formulations include Rifalazil microgranules of a size which is poorly absorbed, which are coated with a gastro-resistant film which dissolves in the colon to release the antibiotic only in the intestinal tract. Ideally, the microgranules in the formulation have a high superficial area, to maximize contact between the active ingredient and the intestinal mucous. These formulations allow one to administer the Rifalazil in relatively high doses.

The novel gastro-resistant, e.g., gastrointestinal retentive minimally absorbed Rifalazil formulations takes advantage of the pH difference between the gastric environment (e.g., values from about 1.5 to about 4.0, depending on the state of fast or in presence of meal) and the intestinal lumen (e.g., values from 5.0 to about 7.5, depending of the tracts considered), so that Rifalazil is minimally absorbed in the GI tract, and so that retention occurs in the GI tract.

The microgranules are coated with a gastro-resistant film. The Rifalazil particles can have a very fine particle size, for example, approximately 50% of the particles have a particle diameter between 10 μm and 40 μm. Thus, they are large enough to remain poorly dissolved, but small enough to use in preparation of microgranules.

Ideally, the granules are provided with an enteric coating using fluidized bed technology, which enables one to simultaneously wet-granulate the powder and coat the microgranules with a polymer resistant to the gastric environment (i.e., an enteric coating). This approach minimizes some of the difficulties associated with wet-granulation and microgranule coating.

Suppository administration forms of Rifalazil are also contemplated by the invention. Rifalazil in a poorly absorbed formulation can also be administered by adding same to or mixing same with food for treatment of CDAD patients. Probiotic formulations may be employed for such purpose, including ingredients such as Lactobacillus, which are incorporated in food materials such as yoghurt or added to other meal foods.

Thus, using the processes described herein, one can prepare a drug delivery composition wherein rifalazil is fully coated by an enteric polymer. The particles sizes can be homogeneous, without large clots or very fine powder.

In order to maximize the release of the active ingredient near the intestinal mucous membrane a high pH difference can be employed between the gastric environment, at values from 1.5 to 4.0, depending on the state of fast or in presence of meal, and the intestinal lumen, at values from 5.0 to 7.5 depending of the tracts considered. For this purpose, enteric polymeric materials having the property to solubilize at pH values between 5.0 and 7.5 can be used, to include: methacrylic acid copolymers with an acrylic or methacrylic ester like methacrylic acid ethylacrylate copolymer (1:1) and methacrylic acid methylmethacrylate copolymer (1:2), polyvinyl acetate phthalate, hydroxypropyl cellulose acetate phthalate and cellulose acetate phthalate, and products available on the market under the trademarks KOLLICOAT®, EUDRAGIT®, AQUATERIC®, AQOAT®.

In the fluidized bed coating step, the film coating, dissolved in organic solvents or suspended in water, is applied by spraying on powders or granules maintained in suspension with air in fluidized bed systems. Representative organic solvents include methylene chloride, methyl alcohol, isopropyl alcohol, acetone, tri-ethyl acetate and ethyl alcohol. Alternatively, the polymeric gastro-resistant material can be applied suspended in water.

Other excipients with anti-agglomerative properties can also be used. Examples include talc; plasticizing materials, like acetylated glycerides, diethylphthalate, propylene glycol and polyethylene glycol; surfactants like polysorbate and polyoxyethylenate esthers, anti-foaming agents, as well as anti-sticking agents.

The gastro-resistant Rifalazil microgranules can then be used directly to fill capsules or can be mixed with excipients and sweetener enhancers, e.g., in an aqueous suspension administration. The gastro-resistant Rifalazil microgranules can also be directly used for tablet preparation through direct compression technology by adding conventional vehicles or carriers.

The microgranules remain insoluble in the stomach (e.g., at a range of pH between about 1.5 and about 4.0) and soluble in the intestine (e.g., at higher pH, for example between about 5.5 and about 7.5.), to administer high doses of Rifalazil, targeting maximum release in the intestine, while maximizing contact with the intestinal mucous membrane due to the high superficial area of the microgranules.

The microgranules can typically range in size between about 1 micron to about 900 microns in diameter, or more preferably from between about 10 microns to about 500 microns in diameter. The gastro-resistance can be obtained using any material insoluble at pH values ranging between about 1 to about 4.9, from about 1.4 to about 4.2, or from about 1.5 and about 4.0. Suitable polymers may also be soluble at pH values ranging from between about 5.0 to about 7.0, 5.0 to about 7.5, or 5.0 and about 7.7 and above.

Polymeric materials used in the gastro-resistant Rifalazil formulations solubilize, as discussed above, at pH values consistent with the intestinal lumen, for example, from between about 4.9 and about 7.7, and can be used as gastro-resistant, entero-soluble coatings for drug release in the intestine when desired.

Examples of suitable polymeric materials include, for example, acrylic polymers, methacrylic acid copolymers with an acrylic or methacrylic ester (e.g., methacrylic acid ethylacrylate copolymer (1:1) and methacrylic acid methylmethacrylate copolymer (1:2), polyvinyl acetate phthalate, hydroxypropyl cellulose acetate phthalate and cellulose acetate phthalate), as well as cellulose acetate phthalate, hydroxypropyl methylcellulose phthalate, polyvinyl acetate phthalate. Commercially available products include, for example, KOLLIKOAT®, EDRAGIT® (e.g., EUDRAGIT 40), AQUATERIC®, AQOAT®. The enteric materials, which are soluble at higher pH values, are frequently used for colon-specific delivery systems and are employable in the gastro-resistant Rifalazil formulations described herein. The enteric polymers used can also be modified by mixing with other coating products that are not pH sensitive. Examples of such coating products include, for example, the neutral methacrylic acid esters with a small portion of trimethylammonioethyl methacrylate chloride, sold currently under the trade names EUDRAGIT® and EUDRAGIT® RL; a neutral ester dispersion without any functional groups, sold under the trade names EUDRAGIT® NE30D and EUDRAGIT® NE30, EUDRAGIT® 40; polysaccharides, like amylose, chitosan, chondroitin sulfate, dextran, guar gum, inulin and pectin; and other pH independent coating products.

The polymer in various embodiments is from between about 5% and about 75% of the weight of the microgranule. In other embodiments, the polymer is from between about 10% and about 60%, 20% and about 55%, about 30% to about 80%, or 25% and about 50% of the weight of the microgranule. The weight percent of the polymer to the weight of the microgranule can depend, in part, on the polymer used, the temperature of the polymer, the formulation (e.g., bag, pill, capsule, etc.), and the pH at which the polymer is soluble.

The gastro-resistant Rifalazil microgranules may further comprise one or more of diluents, plasticizers, anti-agglomeratives, anti-sticking, glidants, anti-foam surfactants, or coloring substances. These, along with other polymers and coatings (e.g., protective coatings, over-coatings, and films) are more fully described below.

Suitable ingredients can be incorporated into the coating formula such as plasticizers, which include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxydized tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. Other various layers, as recognized by one of skill in the art are also envisioned. The amount of plasticizer used in the polymeric material typically ranges from about 10% to about 50%, for example, about 10, 20, 30, 40, or 50%, based on the weight of the dry polymer. Optional modifying components of a protective layer which can be used over the enteric or other coatings include a water penetration barrier layer (semi-permeable polymer) which can be successively coated after the enteric or other coating to reduce the water penetration rate through the enteric coating layer and thus increase the lag time of the drug release. Coatings commonly known to one skilled in the art can be used for this purpose by coating techniques such as fluid bed coating using solutions of polymers in water or suitable organic solvents or by using aqueous polymer dispersions. For example, useful materials include cellulose acetate, cellulose acetate butyrate, cellulose acetate propionate, ethyl cellulose, fatty acids and their esters, waxes, zein, and aqueous polymer dispersions such as EUDRAGIT® RS and RL 3OD, EUDRAGIT® NE 3OD, EUDRAGIT® 40, AQUACOAT®, SURELEASE®, cellulose acetate latex. Combinations of the polymers and hydrophilic polymers such as hydroxy ethyl cellulose, hydroxypropyl cellulose (KLUCEL®, Hercules Corp.), hydroxypropyl methylcellulose (METHOCEL®, Dow Chemical Corp.), polyvinylpyrrolidone may also be used.

Anti-foaming agents can also be included in the formulations. In one embodiment, the anti-foaming agent is simethicone. The amount of anti-foaming agent used typically comprises from 0% to 0.5% of the final formulation. Other agents can be added to improve the processability of a sealant or barrier layer. Such agents include, for example, talc, colloidal silica, polyvinyl alcohol, titanium dioxide, micronized silica, fumed silica, glycerol monostearate, magnesium trisilicate, and magnesium stearate, or a mixture thereof.

The amount of polymer to be used in the gastro-resistant formulations is typically adjusted to achieve the desired drug delivery properties, including the amount of drug to be delivered, the rate and location of drug delivery, the time delay of drug release, and the size of the multiparticulates in the formulation. The combination of all solid components of the polymeric material, including co-polymers, fillers, plasticizers, and optional excipients and processing aids, typically provides about 1% to about 50% weight of the core.

The resulting microgranules can be directly compressed in tablet after having mixed with appropriate excipients such as diluents such as dicalcium phosphate, calcium sulphate, cellulose, microcrystalline cellulose (AVICEL®), hydroxypropyl methyl cellulose, corn starch, lactose, kaolin, mannitol, sodium chloride, dry starch; binders such as starch, gelatine, sugars as sucrose, glucose, dextrose, lactose, synthetic gum, sodium alginate, carboxymethyl cellulose, methylcellulose, polyvinylpyrrolidone, polyethylene glycol, ethylcellulose, water, waxes, alcohol; lubricants such as talc, magnesium stearate, calcium stearate, stearic acid, hydrogenated vegetable, oils, polyethylenglycole; glidants such as colloidal silicon dioxide, talc; disintegrants such as corn and potato starch, croscarmelose, crospovidone, sodium starch glycolate, colouring agents, sweeteners such as sucrose, sorbitol, mannitol, saccharine, acesulfame, neohesperedine.

Conventional technology and apparatus known to expert-of-art of tablet preparation can be applied. The gastro-resistant microgranules are mixed with the above mentioned excipients in a suitable apparatus like a biconical mixer or V mixer for the time necessary to obtain the homogeneity of the gastroresistant microgranules inside the mixture.

The granules have good properties in respect of ability to flow freely, cohesiveness and lubrication, therefore the ratio between gastroresistant microgranules and excipients is between 1:0.2 and 1:0.05, preferably between 1:0.15 and 1:0.1. The obtained mixture can be pressed in order to obtain, using a suitable punch, tablets containing a quantity of Rifalazil, e.g., between 50 mg and 600 mg, preferably between 100 mg and 500 mg.

As described above, the favorable properties of Rifalazil gastro-resistant microgranules allow achieving a suitable blend for direct compression with the addition of minimal quantity of excipients.

Tablets can be successively coated with a conventional hydrophilic film to achieve taste-masking properties and improve appearance. Suitable materials in specific embodiments include, without limitation, hydroxyethyl cellulose, hydroxypropyl cellulose (KLUCEL®, Hercules Corp.), hydroxypropyl methylcellulose (METHOCEL®, Dow Chemical Corp.), and polyvinylpyrrolidone.

The tablets can themselves be film-coated using techniques well known to those of skill in the art. Typically, the coatings include cellulose polymers such as hydropropylcellulose hydromethylcellulose, and hydropropyl-methylcellulose. Alternatives to the cellulose ethers include certain acrylics, such as methacrylate and methylmethacrylate copolymers.

The polymers can be used as solutions, utilizing either an aqueous or an organic solvent-based system. Incorporating a plasticizer enables the flexibility of the coating film to be improved; by addition of plasticizers, the risk of film cracking is reduced, and the adhesion of the film to the substrate is improved. Examples of typical plasticizers include glycerin, propylene glicol, polyethylene glycols, triacetin, acetylated monoglycerides, citrate esthers and phtalate esthers. Colorants can be used to improve the appearance of the product. Water-soluble and/or organic solvent-soluble dyes can be used, e.g., albumin lake, titanium dioxide, and iron oxide. Finally, stabilizers such as EDTA can be added to the coating.

Formulations and Dosages for Combination Therapies

Rifalazil can be administered to a subject having antibiotic associated bacterial diarrhea or an infection of C. difficile in conjunction with one or more additional antibiotics. Rifalazil can be administered before, during, or after administration of the additional antibiotics, or any combination thereof. If desired, the administration of Rifalazil can be continued while the additional antibiotic is being administered.

Exemplary antibiotics that can be administered in the methods of the invention are .beta.-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); .beta.-lactamase inhibitors (e.g., clavulanate, sulbactam, and tazobactam); aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); lipopetides (e.g., daptomycin); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); rifamycins (e.g., rifampicin, rifabutin, rifapentine, and rifaximin); metronidazole; garenoxacin; ramoplanin; faropenem; polymyxin; tigecycline, AZD2563; CBR-2092 (Cubre Pharmaceuticals) and trimethoprim.

These antibiotics can be used in the dose ranges and formulations currently known and used for these agents. Different concentrations may be employed depending on the clinical condition of the subject, the goal of therapy (treatment or prophylaxis), the anticipated duration, and the severity of the C. difficile or other infection. Additional considerations in dose selection include the type of infection, age of the subject (e.g., pediatric, adult, or geriatric), general health, and comorbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. M H Beers et al., Merck & Co.) and Physicians' Desk Reference 2003 (57.sup.th Ed. Medical Economics Staff et al., Medical Economics Co., 2002).

In one example, Rifalazil is administered in combination with vancomycin. Either the Rifalazil or the vancomycin or both may be given daily (e.g., once, twice, three times, or four times daily) or less frequently (e.g., once every other day, once every two days, once every three days, once or twice weekly, or monthly). Typical daily dosages for vancomycin range from 20 mg to 2 gm, preferably 125 mg to 2 gm, or 500 mg to 2 gm, but it may be administered in any higher tolerated amounts as necessary. Daily dosages of vancomycin can be distributed over one to four doses. Exemplary daily oral dosages include from 500 mg to 2 gm distributed over one to four doses for adult subjects and 40 mg/kg distributed over one to four doses for pediatric subjects. Intravenous administration can be given as a one-time bolus per 24-hour period, or for any subset of time over the 24-hour period (e.g., half an hour, one hour, two hours, four hours, or up to 24 hours).

For combination therapy, the Rifalazil and the additional antibiotic can be administered simultaneously or sequentially. For sequential administration, the Rifalazil can be administered before, during, or after administration of the additional antibiotic, or any combination thereof. In one example, vancomycin is administered for five days and Rifalazil is administered as a single dose on the sixth day. In another example, vancomycin and Rifalazil are administered simultaneously on day one followed by administration of vancomycin for an additional six days. These examples are provided to illustrate two potential combinations for sequential therapy. They are not intended to limit the invention in any way.

For combination therapy, the dosage and the frequency of administration of each component of the combination can be controlled independently. For example, one of the compounds (i.e., Rifalazil or the additional antibiotic) may be administered three times per day, while the second compound may be administered once per day. The compounds may also be formulated together such that one administration delivers both compounds.

The invention contemplates the use in combination of a therapeutic agent selected from among Rifalazil and Rifalazil derivatives, with any of the following: antiperistaltic agents, narcotic analgesics, and loperamide (Imodium), and the use of Rifalazil or Rifalazil derivative will compensate the exacerbation activities of the other therapeutic agents to the CDAD disease.

The invention further contemplates the use in combination of Rifalazil and Rifalazil derivatives with therapeutics (including antibiotics) used and needed to be maintained in use to treat life threatening diseases despite the fact that they exacerbate the CDAD disease, with the use of Rifalazil (or Rifalazil derivatives) compensating the CDAD effects of the therapeutic agents.

The invention further contemplates the use of Rifalazil and Rifalazil derivatives for treatment of nosocomial infections, using GI retentive minimally absorbed formulations of Rifalazil and Rifalazil derivatives, in prevention or other therapeutic approaches, alone or in combination with other therapeutics.

A further aspect of the invention relates to the use of Rifalazil and Rifalazil derivatives in combination with probiotic bacteria that are effective for treatment or prophylaxis of nosocomial infection.

Clostridium difficile outbreaks, antibiotic-associated diarrhoea (AAD) and rota viral outbreaks in paediatric patients may be treated with such compositions of the invention.

Pharmaceutical Packages

The invention also features a pharmaceutical pack comprising (i) Rifalazil in an amount effective to treat a subject having antibiotic-associated bacterial diarrhea or an infection of C. difficile; and (ii) instructions for administering the Rifalazil to a subject for treating or preventing a C. difficile infection. Desirably, the Rifalazil is in unit amounts, such as between 0.01 and 1000 mg (e.g., between 1 and 100 mg, or between 1 and 50 mg, or between 1 and 25 mg, or between 1 and 5 mg), and is present in amounts sufficient to treat for at least 1, 3, 5, 7, 10, 14, 21, or 31 days. The pharmaceutical pack of the invention can further comprise one or more antibiotics. Preferred examples of the additional antibiotic include metronidazole, gentamicin, daptomycin, azithromycin, quinupristin, dalfopristin, linezolid, teicoplanin, ciprofloxacin., and vancomycin. Typical dosages for vancomycin range from 20 to 2000 mg, preferably from 125 to 2000 mg.

Exemplary additional antibiotics that can be administered in the methods of the invention or included in the pharmaceutical pack of the invention are beta-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); .beta.-lactamase inhibitors (e.g., clavulanate, sulbactam, and tazobactam); beta-lactamases, specifically designed to inactivate residual amounts of antibiotics in the patient's gastrointestinal tract, after parenteral administration of beta-lactam antibiotics for serious infections; aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); lipopetides (e.g., daptomycin); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); rifamycins (e.g., rifampicin, rifabutin, rifapentine, and rifaximin); metronidazole; garenoxacin; ramoplanin; faropenem; polymyxin; tigecycline, AZD2563; REP3123, OPT-80 and trimethoprim, C. difficile toxin-specific inhibitors (e.g., tolevamer).

In an era of increasing concern about the overuse of antibiotics and the emergence of antibiotic resistance and “superbugs,” products designed to bind and remove from the body toxins released by C. difficile that damage the large intestine, like tolevamer (Genzyme), have the potential not only to treat CDAD, but also to reduce its rate of recurrence through a non-antibiotic mechanism of action that does not harm the normal intestinal bacteria that provide protection against C. difficile.

OPT-80, formerly known as PAR-101 or Difimicin, is a narrow spectrum antibiotic in development to treat CDAD. OPT-80, which is cidal against (i.e., kills) C. difficile, is unlike the current FDA-approved treatment which only inhibits bacteria growth. OPT-80 has shown selective activity against C. difficile while leaving the healthy intestinal flora intact. This selective activity, while eliminating the infection, may have utility to preserve the natural balance of flora in the GI tract

REP3123 is a novel inhibitor of methionyl tRNA synthetase, a protein that is essential for protein biosynthesis in bacteria. It competitively binds to the bacteria RNA at the active site of the biosynthesis.

### III. Methods of Treatment

Being virtually nonabsorbed, Rifalazil's bioavailability within the GI tract is rather high, with intraluminal and fecal drug concentrations that largely exceed the minimal inhibitory concentration values observed in vitro against a wide range of pathogenic organisms. The GI tract represents, therefore, the primary therapeutic target and GI infections the main indication.

The pathogenic role of gut bacteria in several organic and functional GI diseases has been increasingly recognized as being at least partially responsible for hepatic encephalopathy, small intestine bacterial overgrowth, inflammatory bowel disease and colonic diverticular disease.

The compositions can also be used to treat irritable bowel syndrome, chronic constipation, and Clostridium difficile infection (CDAD infection), as well as for bowel preparation before colorectal surgery.

Rifalazil is also active against Helicobacter pylori, and can be used to eradicate Helicobacter pylori.

Oral administration of Rifalazil can eliminate enteric bacteria, and can be employed to achieve selective bowel decontamination in acute pancreatitis, liver cirrhosis (thus preventing spontaneous bacterial peritonitis), and nonsteroidal anti-inflammatory drug (NSAID) use (lessening in that way NSAID enteropathy).

Because Rifalazil has poor solubility, when administered according to the teachings of the invention, it will have little activity outside the enteric area, and thus will minimize both antimicrobial resistance and systemic adverse events.

Treatment of CDAD

Clostridium difficile is an anaerobic Gram-positive, spore-forming toxigenic bacillus, infrequently found in significant numbers in the colon of humans. However, because it is refractory to a number of antimicrobial agents and is endemic in hospitals and nursing homes, it can appear when the normal bacterial flora of the colon is suppressed, most often after treatment with broad-spectrum antibacterial agents. Under these circumstances, C. difficile can cause severe diseases, known as antibiotic-associated diarrhea and pseudomembranous colitis.

Traditional treatments for these disorders include metronidazole and oral vancomycin. Currently, however, the use of vancomycin is being actively discouraged because, particularly in an oral form, it selects for a new class of highly resistant intestinal organisms, vancomycin-resistant enterococci (VRE), which can cause fatal, untreatable infections at other body sites. Metronidazole is not active against enterococci, so its use may also contribute to selection of VRE in the colon. The relapse rate for C. difficile disease is very high, about 20%; it is thought that this may be related to the formation of spores, which are difficult to eradicate.

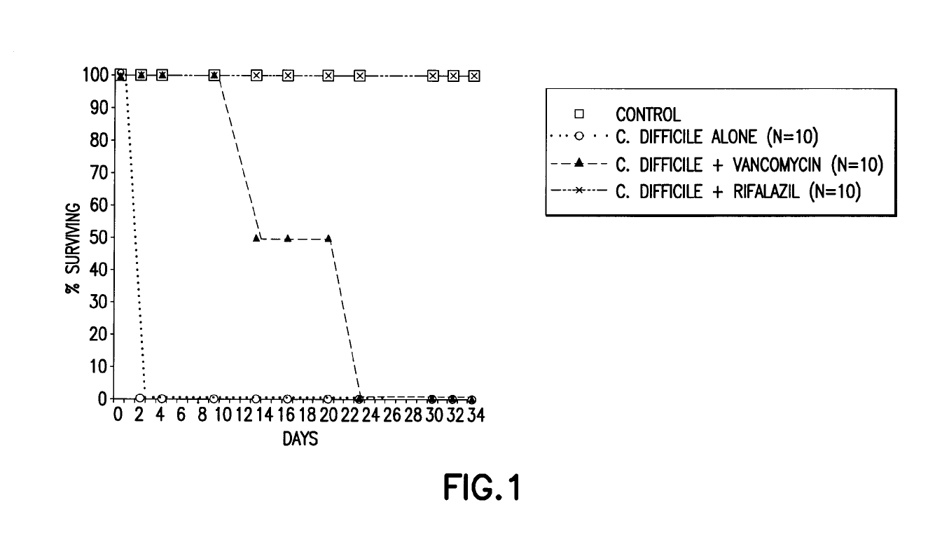

In in vivo animal experiments using microgranulated Rifalazil, which provides for systemic administration of Rifalazil, CDAD was effectively treated, and no relapses were observed (See FIG. 1). However, the treatments required relatively high doses of Rifalazil, albeit in a microgranulated formulation. Correspondingly high doses are not necessarily desirable in humans.

In one embodiment, the methods described herein are directed to the use of a poorly absorbed form of Rifalazil, administered locally to colon, but not available systemically, to treat the CDAD infection. The methods involve treating CDAD by maintaining an active concentration of Rifalazil in the colon for a relatively long period of time. That is, by minimizing the systemic circulation of Rifalazil, and, ideally, by delivering the Rifalazil to the colon in a drug delivery composition that is specific for colonic administration, the Rifalazil remains in the colon for a suitable period of time to treat CDAD.

In one embodiment, a small portion of the dosage of Rifalazil is absorbed systemically, for example, by including microparticulate forms of Rifalazil in combination with the larger particle forms, so the microparticulate forms can travel systemically, and recirculate in the colon at a later time. That is, Rifalazil is eliminated predominantly via biliary excretion. Based on the long half-life of Rifalazil, by re-circulating a portion of the rifalazil to the colon, one can prevent relapses of the disorder, should any of the bacteria survive the initial presentation of Rifalazil in the colon.

Co-Administration with Vancomycin or Other AntiBiotics

In another embodiment, rather than, or in addition to, including Rifalazil in absorbable form (i.e., microparticulate form), one can co-administer oral Vancomycin. The co-administration of Rifalazil can minimize the development of vanco-Resistant Enetrococcis (VREs) and VRSA (Vanco Resistant Staph aureus).

In certain embodiments of the invention, the method includes administering Rifalazil and vancomycin simultaneously or sequentially. Rifalazil and vancomycin can be administered within fourteen days of each other, or within five days, three days, or within twenty-four hours of each other. If desired, either Rifalazil or vancomycin, or both can be administered orally. Preferred dosages for vancomycin in specific embodiments can range from 20 to 2000 mg per day, preferably from 125 to 2000 mg per day, most preferably from 500 to 2000 mg per day.

The dosage of Rifalazil in various embodiments can range from 0.01 mg to 1000 mg. The dosage of Rifalazil is e.g., normally about 1 to 1000 mg (desirably about 1 to 100 mg, more desirably about 1 to 50 mg, and even more desirably about 1 to 25 mg). The Rifalazil may be given daily (e.g., once, twice, three times, or four times daily) or less frequently (e.g., once every other day, once or twice weekly, or monthly). Rifalazil is administered for a length of time sufficient to treat the subject. Treatment may be for 1 to 31 days, desirably 1 to 21 days, 1 to 14 days or even 1, 3, 5, or 7 days. If desired, treatment can continue for up to a year or even for the lifetime of the subject. In one example, Rifalazil is administered at an initial dose of between 5 and 100 mg, followed by subsequent doses of between 1 and 50 mg for 3 to 7 days. A single dose of Rifalazil (e.g., in a dosage of between 1 and 100 mg) can also be employed in the method of the invention. The Rifalazil may be administered orally, intravenously, subcutaneously, or rectally, though oral administration in a drug delivery vehicle designed to deliver its contents to the colon is particularly preferred.

The method can be employed as an initial treatment of a subject having or being at risk for developing antibiotic-associated bacterial diarrhea or an infection of C. difficile, or it may be employed to treat subjects for whom the initial treatment (e.g., with metronidazole, vancomycin, rifampicin, rifabutin, rifapentine, and rifaximin) has failed to fully treat the antibiotic-associated bacterial diarrhea or an infection of C. difficile. The method may be employed, for example, when the subject is colonized with C. difficile organisms that are resistant to one or more of metronidazole, vancomycin, rifampicin, rifabutin, rifapentine, and rifaximin.

If desired, Rifalazil can be administered with one or more additional antibiotics or with an agent that binds toxin A or toxin B (e.g., the non-absorbed toxin binding polymer GT160-246; U.S. Pat. No. 6,270,755). Preferred examples of additional antibiotics are metronidazole, gentamicin, daptomycin, azithromycin, quinupristin, dalfopristin, linezolid, teicoplanin, ciprofloxacin, and vancomycin.

The following examples are shown to illustrate, but not to limit the present invention.

### EXAMPLES

### Example 1

### Animal Models of C. Difficile-Associated Disease

Optimal dosages and formulations of Rifalazil alone, or in combination with a second drug compound, can be determined using standard animal models known in the art. One example of an animal model for C. difficile associated disease is the Golden Syrian hamster. To determine the optimal dosage regimen of Rifalazil, Golden Syrian hamsters are injected subcutaneously with clindamycin phosphate (10 mg/kg) followed, 24 hours later, by oral gavage with 10⁵ colony forming units (CFU) of C. difficile. Antibiotic treatment is then administered orally, either simultaneously or 24 hours after C. difficile administration Animals are monitored for survival, weight variations, identification of C. difficile toxins in cecal content, and histologic damage to ceca as compared to animals treated with a prophylactic protocol using standard methods known in the art (see, for example, Anton P. M. et al., Abstract ID No. 102471, Publishing ID No. T1741, presented at the American Gastroenterological Association Meeting, May 17-22, 2003; Anton P. M. et al., Gastroenterology 124:A558, 2003).

### Example 2

### CDAD Treatment in Animals Using Rifalazil

Hamsters were infected with C. difficile, and at the time of infection, were also treated with vancomycin (50 mg/kg) or Rifalazil (2 mg/kg). All animals treated with Rifalazil survived, whereas those treated with vancomycin initially appeared to have been treated, but eventually succumbed to the infection.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in microbiology or related fields are intended to be within the scope of the invention.

1. A method for treating a subject having an infection of Clostridium difficile, said method comprising administering to said subject an effective amount of Rifalazil in a poorly absorbed form, wherein the average particle size of the Rifalazil is greater than about 10 μm.

2. The method of claim 1, wherein said Rifalazil is administered in an amount between 0.01 and 1000 mg/day.

3. The method of claim 2, wherein said Rifalazil is administered in an amount between 1 and 100 mg/day.

4. The method of claim 3, wherein said Rifalazil is administered in an amount
between 1 and 50 mg/day.

5. The method of claim 4, wherein said Rifalazil is administered in an amount between 5 and 25 mg/day.

6. The method of claim 1, wherein said Rifalazil is administered for one to fourteen days.

7. The method of claim 6, wherein said Rifalazil is administered for three to seven days.

8. The method of claim 1, wherein said Rifalazil is administered as a single dose.

9. The method of claim 8, wherein the dose is administered for two consecutive days.

10. The method of claim 8, wherein the dose is administered for three consecutive days.

11. The method of claim 1, wherein said Rifalazil is administered at an initial dose of between 5 and 100 mg, followed by subsequent doses of between 0, 1 and 50 mg for three to seven days.

12. The method of claim 1, wherein said infection of Clostridium difficile comprises a strain of Clostridium difficile that is resistant to one or more antibiotics selected from the group consisting of vancomycin, macrolide, ansamycin, rifampicin, rifabutin, rifapentine, rifaximin, and metronidazole.

13. The method of claim 1, wherein said rifalazil is administered in the form of a drug delivery composition for oral administration, and colonic delivery, of the Rifalazil.

14. The method of claim 1, further comprising administering to said subject one or more agent that binds Clostridium difficile toxin A or toxin B.

15. The method of claim 1, further comprising administering to said subject one or more antibiotics selected from the group consisting of beta-lactams, betalactamase inhibitors, aminoglycosides, tetracyclines, lipopetides, macrolides, ketolides, lincosamides, streptogramins, sulphonamides, oxazolidinones, quinolones, rifamycins, glycopeptides, metronidazole, garenoxacin, ramoplanin, faropenem, polymyxin, tigecycline, AZD2563, and trimethoprim.

16. The method of claim 15, wherein said quinolone is ciprofloxacin.

17. The method of claim 15, wherein said rifamycin is selected from the group consisting of rifampicin, rifabutin, rifapentine, and rifaximin. 18. The method of claim 15, wherein said antibiotic is metronidazole.

19. The method of claim 15, wherein said glycopeptide is vancomycin.

20. The method of claim 19, wherein said Rifalazil and vancomycin are administered simultaneously.

21. The method of claim 20, wherein the Rifalazil and vancomycin are administered in a fixed formulation, or in separate formulations, or combined with a ligand.

22. The method of claim 19, wherein said Rifalazil and vancomycin are administered sequentially.

23. The method of claim 19, wherein said Rifalazil and vancomycin are administered within fourteen days of each other.

24. The method of claim 19, wherein said vancomycin is administered in an amount between 125 and 2000 mg per day.

25. A method of treating a subject having an infection of Clostridium difficile, said method comprising administering to said subject a composition comprising Rifalazil, in a form which is poorly solubilized, along with vancomycin, in a separate or a fixed formulation.

26. The method of claim 25, wherein said composition is suitable for oral administration.

27. The method of claim 25, wherein said Rifalazil is in a unit dosage amount between 0.01 and 100 mg, and said vancomycin is in a unit dosage amount between 125 and 2000 mg.

28. The method of claim 25, wherein said Rifalazil is in a unit dosage amount between 1 and 50 mg, and said vancomycin is in a unit dosage amount between 500 and 2000 mg.

29. The method of claim 25, wherein said Rifalazil is in a unit dosage amount between 1 and 25 mg, and said vancomycin is in a unit dosage amount between 500 and 2000 mg.

30-59. (canceled)

60. A pharmaceutical pack comprising
(i) Rifalazil in an amount effective to treat a subject having an infection of Clostridium difficile, wherein the Rifalazil is in a form that is poorly absorbed systemically; and
(ii) instructions for administering said Rifalazil to said subject for treating a Clostridium difficile infection.

61. The pharmaceutical pack of claim 60, wherein said Rifalazil is in a unit dosage amount between 0.01 and 100 mg.

62. The pharmaceutical pack of claim 60, wherein said Rifalazil is in an amount between 1 and 50 mg.

63. The pharmaceutical pack of claim 62, further comprising one or more antibiotics selected from the group consisting of beta.-lactams, beta-lactamase inhibitors, aminoglycosides, tetracyclines, lipopetides, macrolides, ketolides, lincosamides, streptogramins, sulphonamides, oxazolidinones, quinolones, rifamycins, glycopeptides, metronidazole, garenoxacin, ramoplanin, faropenem, polymyxin, tigecycline, AZD2563, and trimethoprim.

64. The pharmaceutical pack of claim 63, wherein said quinolone is ciprofloxacin.

65. The pharmaceutical pack of claim 63, wherein said rifamycin is selected from the group consisting of rifampicin, rifabutin, rifapentine, and rifaximin.

66. The pharmaceutical pack of claim 63, wherein said glycopeptide is vancomycin.

67. The pharmaceutical pack of claim 66, wherein said vancomycin is in an amount between 125 and 2000 mg.

68. The pharmaceutical pack of claim 66, wherein said vancomycin is in an amount between 500 and 2000 mg.

69-70. (canceled)

71. A method of treatment of a nosocomial infection, comprising administration to a subject in need thereof, of Rifalazil or a Rifalazil derivative, in combination with administration of another therapeutic agent with which Rifalazil or a Rifalazil derivative compensates exacerbation by the therapeutic agent of CDAD disease.

72. The method of claim 71, wherein said Rifalazil is administered on each of two successive days.

73. The method of claim 71, wherein said Rifalazil is administered on each of three successive days.

74. The method of claim 71, wherein the agent is tolevamer.

75-78. (canceled)
